(19) United States
(12) Patent Application Publication
(10) Pub. No.: US 2006/0159716 A1
(43) Pub. Date: Jul. 20, 2006

(54) Cosmetic emulsion comprising solid particles, method for preparing cosmetic emulsion, and method of using cosmetic emulsion

(76) Inventors: Agnes Themens, Bourg la Reine (FR); Pascal Arnaud, L'Hay les Roses (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/305,145

(22) Filed: Dec. 19, 2005

Related U.S. Application Data
(60) Provisional application No. 60/638,120, filed on Dec. 23, 2004.

(30) Foreign Application Priority Data
Dec. 17, 2004 (FR) 4 53045

(51) Int. Cl. A61K 8/89; A61K 8/36

(52) U.S. Cl. 424/401

(57) ABSTRACT

The present disclosure relates to a cosmetic composition for making up or caring for the skin in the form of an emulsion comprising an aqueous phase and an oily phase, comprising a dispersion of wax particles with a mean size by volume of less than or equal to 1 μm in the aqueous phase and, dispersed in the oily phase, particles with a mean size by volume of less than or equal to 15 μm. This present disclosure further relates to a process for treating skin with the compositions described herein.

This application claims benefit of U.S. Provisional Application No. 60/638,120, filed Dec. 23, 2004, the contents of which are incorporated herein by reference. This application also claims benefit of priority under 35 U.S.C. § 119 to French Patent Application No. 04 53045, filed Dec. 17, 2004, the contents of which are also incorporated herein by reference.

The present disclosure relates to a cosmetic composition for making up or caring for the skin, including the lips, in emulsion form, a method for making such composition, and a method of use thereof.

The cosmetic composition according to the present disclosure can be a foundation, such as, for example, a foundation to be applied to the face or the neck, a concealer, a tinted cream, a composition for making up the body, an eye shadow, a blusher or a lipstick.

The cosmetic composition can be a product for caring for the skin, such as a care base for the skin, a care cream (e.g., day cream, night cream, anti-wrinkle cream), a make-up base or a composition for caring for the lips (e.g., lip balm).

During the aging process, various signs appear on the skin, being reflected, for example, by modification of the cutaneous functions and structure of the skin. The main clinical signs of cutaneous aging include the appearance of fine lines and deep wrinkles, which increase with age. A disruption of the “grain” of the skin can be observed, that is to say that the microrelief is less uniform and exhibits an anisotropic nature.

It is known to treat these signs of aging by using cosmetic or dermatological compositions comprising active principles capable of combating aging. These active principles may act on the wrinkles by removing dead cells from the skin and/or by accelerating the process of cell replacement. However, these active principles can exhibit the disadvantage of only being effective in the treatment of wrinkles after a certain application time. In point of fact, it is increasingly desired to obtain an immediate effect of the active principles used.

Other known methods for concealing imperfections of the skin include using compositions comprising fillers said to have a soft-focus effect, as is disclosed in European Patent Application No. EP 1 099 437 A. These compositions, by an optical effect, can tone down cutaneous imperfections or blemishes, such as blotches, wrinkles or fine lines.

However, the results obtained with these compositions can often be inadequate in effectively toning down, or indeed even masking, the most pronounced wrinkles.

In addition, the solid particles, such as fillers or pulverulent coloring materials, often present in compositions for making up the skin can exhibit the disadvantage of becoming concentrated in the wrinkles, in particular the deep wrinkles, thus accentuating the unevenness of the skin. The application of these compositions to particularly wrinkled skin (for example, mature skin) can result in a make-up which shows or discloses the wrinkles.

There thus exists a need for a composition for making up or caring for the skin which can make it possible to obtain satisfactory concealment of the imperfections or blemishes of the skin, such as wrinkles, fine lines or blotches.

The inventors have discovered, surprisingly, that it is possible to obtain such a composition by the introduction, into the oily phase and into the aqueous phase of an emulsion, of particles chosen according to their size and/or their chemical nature.

Accordingly, the present disclosure relates to a cosmetic composition for making up or caring for the skin in the form of an emulsion comprising an aqueous phase and an oily phase, wherein the aqueous phase comprises a dispersion of at least one wax particles with a mean size by volume of less than or equal to 1 μm, and the oily phase comprises a dispersion of particles with a mean size by volume of less than or equal to 15 μm.

As used herein, the terms “an aqueous phase” and “an oily phase” are understood to mean “at least one aqueous phase” and “at least one oily phase,” respectively.

The present disclosure further relates to a cosmetic composition for making up or caring for the skin in the form of an emulsion comprising an aqueous phase and an oily phase, wherein the emulsion comprises a dispersion of at least one compound in the form of particles with a mean size by volume of less than or equal to 1 μm in the aqueous phase and a dispersion of particles of the same at least one compound with a mean size by volume of less than or equal to 50 μm in the oily phase.

According to one embodiment of the present disclosure, the particles with a mean size by volume of less than or equal to 1 μm dispersed in the aqueous phase may be chosen from waxes, inorganic compounds and mixtures thereof.

The present disclosure still further relates to a cosmetic composition for making up or caring for the skin in the form of an emulsion comprising an aqueous phase and an oily phase, wherein the emulsion comprises particles with a mean size by volume of less than or equal to 1 μm chosen from waxes, silica particles and mixtures thereof dispersed in the aqueous phase and particles chosen from wax powders, silica powders and mixtures thereof dispersed in the oily phase.

According to another embodiment of the present disclosure, the composition may comprise wax particles with a mean size by volume of less than or equal to 1 μm dispersed in the aqueous phase and a wax powder in dispersion in the oily phase.

The present disclosure also relates a cosmetic composition for making up or caring for the skin in the form of an emulsion comprising an aqueous phase and an oily phase, wherein the emulsion comprises particles of at least one material possessing a tightening effect with a mean size by volume of less than or equal to 1 μm dispersed in the aqueous phase and particles with a mean size by volume of less than or equal to 15 μm dispersed in the oily phase.

The present disclosure still further relates to a non-therapeutic process for making up or caring for the skin comprising applying to the skin a composition as defined above.

The present disclosure also relates to the use of a composition as defined above for softening wrinkles and/or giving a firm and toned appearance to the skin.

The present disclosure additionally relates to the use of a composition as defined above for producing a make-up for the skin which softens wrinkles and/or which confers a firm and tonic appearance on the skin.

The mean size by volume of the particles can be measured by various techniques. Mention may be made of light scattering techniques (dynamic and static), Coulter counter methods, measurements by rate of sedimentation (related to the size via Stokes' law) and microscopy. These techniques make it possible to measure a particle diameter and, for some of them, a particle size distribution.

For example, the sizes and size distributions of the particles of the compositions according to the present disclosure may be measured by static light scattering using a commercial particle sizer of MasterSizer 2000 type from Malvern. The data can be processed on the basis of the Mie scattering theory. This theory, accurate for isotropic particles, makes it possible to determine, in the case of nonspherical particles, an “effective” particle diameter. This theory is described, for example, in the work by Van de Hulst, H. C., “Light Scattering by Small Particles,” Chapters 9 and 10, Wiley, New York, 1957.

The particles of the composition are characterized by their mean “effective” diameter by volume D[4,3], defined according to the equation:

$$D [ 4 , 3 ] = \frac{{\sum }_{i} {V}_{i} \cdot {d}_{i}}{{\sum }_{i} {V}_{i}}$$

where Vᵢ is the volume of the particles with an effective diameter dᵢ. This parameter is described, for instance, in the technical documentation of the particle sizer.

The measurements are carried out at 25° C. on a dilute dispersion of particles obtained from the composition in the following way: 1) dilution by a factor of 100 with the predominant solvent of the phase to be analyzed, 2) homogenization of the solution, 3) standing of the solution for 18 hours, 4) recovery of the off-white homogeneous supernatant.

The “effective” diameter is obtained by taking a refractive index of 1.33 for the water and a mean refractive index of 1.42 for the particles.

The particles of wax in the compositions according to the present disclosure can be characterized by a size expressed as a mean “effective” diameter by volume D[4,3] of less than or equal to 1 μm, such as, for example, of less than or equal to 0.75 μm or less than or equal to 0.55 μm.

The composition according to the present disclosure comprises a dispersion of particles of at least one compound in the aqueous phase. The particles of the at least one compound which are dispersed in the aqueous phase can have a mean size by volume of less than or equal to 1 μm (such as, for example, ranging from 0.1 nm to 1 μm), or the particles may have a mean size by volume of less than or equal to 0.5 μm (such as, for example, ranging from 3 nm to 0.5 μm) or particles ranging from 3 nm to 0.3 μm.

The at least one compound in the form of particles dispersed in the aqueous phase of the emulsion according to the present disclosure can be chosen from waxes, inorganic materials and mixtures thereof. In one embodiment, the particles dispersed in the aqueous phase of the emulsion may be chosen from waxes.

According to one embodiment, the composition comprises wax particles with a mean size by volume of less than or equal to 1 μm dispersed in the aqueous phase. Such a dispersion is also referred to as aqueous microdispersion of wax particles.

According to the present disclosure, a wax is understood to mean a lipophilic compound which is solid at ambient temperature (25° C.), which has a reversible solid/liquid change in state and which has a melting point of greater than or equal to 30° C. which can range up to 120° C.

The melting point of the wax can be measured using a differential scanning calorimeter (DSC), for example the calorimeter sold under the name DSC 30 by Metier. A 15 mg sample of product placed in a crucible is subjected to a first rise in temperature ranging from 0° C. to 120° C., at a heating rate of 10° C./minute, is then cooled from 120° C. to 0° C. at a cooling rate of 10° C./minute and, finally, is subjected to a second rise in temperature ranging from 0° C. to 120° C., at a heating rate of 5° C./minute. During the second rise in temperature, the variation in the difference in power absorbed by the empty crucible and by the crucible comprising the sample of product is measured as a function of the temperature. The melting point of the compound is the value of the temperature corresponding to the tip of the peak of the curve representing the variation in the difference in power absorbed as a function of the temperature.

Waxes capable of being used in the microdispersion form in the composition according to the present disclosure may be chosen from waxes which are solid and rigid at ambient temperature of animal, vegetable, mineral or synthetic origin and mixtures thereof. The waxes can exhibit a melting point of greater than or equal to 45° C. (for example, ranging from 45° C. to 120° C.), greater than or equal to 55° C. (for example, ranging from 55° C. to 120° C.), or greater than or equal to 70° C. (for example, ranging from 70° C. to 120° C.).

Waxes within the meaning of the present disclosure may include hydrocarbon, silicone and/or fluorinated waxes optionally comprising ester or hydroxyl functional groups. They may be of natural origin, such as beeswax, which is optionally modified, carnauba, candelilla, ouricury, Japan, cork fibre or sugarcane wax, ceresin, paraffin or lignite waxes, microcrystalline waxes, lanolin wax, montan wax, ozokerites, hydrogenated oils, such as hydrogenated jojoba oil or copolymerization of ethylene, the waxes obtained by the Fischer-Tropsch synthesis, fatty acid esters and glycerides which are solid at 45° C., silicone waxes, such as the alkyl, alkoxy and/or ester derivatives of poly(di)methylsiloxane which are solid at 45° C., having from 10 to 45 carbon atoms, some fatty acids, such as stearic, myristic or behenic acid, and mixtures thereof.

Wax microdispersions are dispersions of colloidal wax particles and are described, for example, in “Microemulsions Theory and Practice,” edited by L. M. Prince, Academic Press (1977), pages 21-32.

Wax microdispersions can be obtained by melting the wax in the presence of a surfactant, and optionally of a portion of the water, followed by gradual addition of hot water with stirring. The intermediate formation of an emulsion of the water-in-oil type, followed by a phase inversion, with final production of a microemulsion of the oil-in-water type, is observed. On cooling, a stable microdispersion of solid colloidal wax particles is obtained. Wax microdispersions can also be obtained by stirring the mixture of wax, surfactant and water using stirring means, such as ultrasound, a high pressure homogenizer or turbine mixers.

The particles of the wax microdispersion may have mean sizes by volume of less than 1 μm (for example, ranging from 0.02 μm to 1 μm), less than 0.5 μm (for example, ranging from 0.05 μm to 0.5 μm), or mean sizes by volume ranging from 0.05 μm to 0.3 μm.

The particles of wax may be composed of a wax or of a mixture of waxes. However, the particles of wax may comprise minor proportions of oily and/or pasty fatty additives, a surfactant and/or a conventional fat-soluble additive/active principle.

According to another embodiment, the composition comprises particles of inorganic compounds which can be chosen from silica and aluminas (such as, for example, boehmite), which have a mean size by volume of less than 1 μm and which are dispersed in the aqueous phase. In a further embodiment, the composition comprises particles of silica having a mean size by volume of less than 1 μm dispersed in the aqueous phase.

According to still another embodiment, the particles dispersed in the aqueous phase can be particles of at least one material having a tightening effect with a mean size by volume of less than or equal to 1 μm.

According to the present disclosure, the term “material having a tightening effect” is understood to mean any agent which produces, at a concentration of 7% in water, a retraction of the isolated stratum corneum, measured with an extensometer, of at least 0.9%, such as more than 1.5%, at 30° C. under a relative humidity of 40%.

The principle of the method consists in measuring the length of a test specimen of stratum corneum isolated from human skin originating from a surgical operation, before and after treatment with the potential tightening agent.

To do this, the test specimen is placed between the two jaws of the device, one of which is fixed and the other of which is movable, in an atmosphere at 30° C. and 40% relative humidity. A pull is exerted on the test specimen and the curve of the force (in grams) as a function of the length (in millimetres) is recorded, the zero length corresponding to the contact between the two jaws of the device. The tangent to the curve in its linear region is subsequently plotted. The intersection of this tangent with the axis of the abscissa corresponds to the apparent length Lo of the test specimen at zero force. The test specimen is subsequently relaxed and then 2 mg/cm² of the test composition (7% solution of the tightening agent under consideration) are applied to the stratum corneum. After drying for 15 minutes, the above stages are again carried out in order to determine the length L, of the test specimen after treatment. The percentage of retraction is defined by the equation: % retraction=100×(L₁−L₀)/L₀. In order to characterize a tightening effect, this percentage has to be negative and the tightening effect becomes greater as the absolute value of the percentage of retraction increases.

Among the materials having a tightening effect that may be used according to the present disclosure, non-limiting mention may be made of colloidal particles of inorganic fillers or alternatively of aqueous microdispersions of wax particles as described above.

According to the present disclosure, the term “colloidal particles” is understood to mean particles in dispersion in an aqueous, aqueous/alcoholic or alcoholic medium, such as, for example, an aqueous medium, having a mean diameter by volume ranging from 0.1 nm to 100 nm, such as, for example, from 3 nm to 30 nm.

As examples of colloidal particles of inorganic fillers that may be used according to the present disclosure, non-limiting mention may be made, inter alia, of silica, cerium oxide, zirconium oxide, alumina, calcium carbonate, barium sulphate, calcium sulphate, zinc oxide and titanium dioxide. Silica is an inorganic filler used according to one embodiment of the present disclosure. Colloidal silica particles are available, for example, in the form of an aqueous dispersion of colloidal silica from Catalysts & Chemicals under the trade names Cosmo S-40 and Cosmo S-50.

In an alternative form, the colloidal particles of inorganic fillers can be mixed colloidal particles comprising at least two different types of metal oxides and composed, for example, of titanium dioxide coated with at least one material, such as titanium dioxide coated with silica.

One example of colloidal particles of inorganic filler can be colloidal particles of silica/alumina composite. The term “silica/alumina composite” is understood to mean silica particles in which aluminium atoms have been partially substituted for silicon atoms.

At a pH of 7, these colloidal particles of silica/alumina composite have a zeta potential of less than −20 mV, such as, for example, less than −25 mV. The measurements are carried out at 25° C. using a Delsa 440SX device from Coulter Scientific Instrument.

Non-limiting mention may be made, as colloidal particles of silica/alumina composites which can be used in the compositions according to the present disclosure, for example, of those sold by Grace under the names of Ludox AM, Ludox AM X 6021, Ludox HSA and Ludox TMA.

Particles having a mean size by volume of less than or equal to 1 μm dispersed in the aqueous phase can be present in an amount ranging from 0.01% to 15% by weight, with respect to the total weight of the composition, such as from 0.5% to 10% by weight, from 1% to 8% by weight, or from 1% to 7% by weight.

The aqueous phase of the emulsion according to the present disclosure can comprise at least one dispersing surfactant which makes it possible to disperse the particles with a mean size by volume of less than or equal to 1 μm.

According to one embodiment of the present disclosure, the surfactant may be chosen from the following compounds:

anionic surfactants, such as, for example, salts of optionally unsaturated fatty acids having, for example, 12 to 18 carbon atoms; alkali metal salts or salts of organic bases of alkyl sulphates and alkylsulphonic acids having 12 to 18 carbon atoms or of alkylarylsulphonic acids, the alkyl chain of which comprises 6-18 carbon atoms; or ether sulphates;

nonionic surfactants, such as, for example, polyalkoxylated and/or polyglycerolated surfactants, including fatty acids or fatty acid amides; alkylphenols or fatty alcohols; esters of fatty acids and of polyols; alkanediols and alkanediol alkyl ethers. Non-limiting mention may also be made of triglycerol alkylcarbamates, oxyethylenated or propoxylated derivatives of lanolin alcohols, lanolin fatty acids, or mixtures thereof; and

cationic surfactants, such as, for example, quaternary ammonium derivatives.

The aqueous phase of the emulsion according to the present disclosure comprises water. The water can be a floral water, such as cornflower water, and/or a mineral water, such as water from Vittel, water from Lucas or water from La Roche Posay, and/or a thermal water. The water can be present in the emulsion according to the present disclosure in an amount ranging from 10% to 80% by weight with respect to the total weight of the composition, such as, for example, ranging from 15% to 60% by weight or ranging from 20% to 50% by weight.

The aqueous phase may also comprise at least one water-miscible (at ambient temperature, 25° C.) organic solvent, such as, for example, monoalcohols having from 2 to 6 carbon atoms, such as ethanol or isopropanol; polyols having, for example, from 2 to 20 carbon atoms, such as those having from 2 to 10 carbon atoms or having from 2 to 6 carbon atoms, such as glycerol, propylene glycol, butylene glycol, pentylene glycol, hexylene glycol, dipropylene glycol or diethylene glycol; glycol ethers (having, for example, from 3 to 16 carbon atoms), such as mono-, di- or tripropylene glycol (C₁-C₄)alkyl ethers or mono-, di- or triethylene glycol (C₁-C₄)alkyl ethers; and mixtures thereof.

The emulsion according to the present disclosure may comprise at least one water-miscible organic solvent, such as, for example, a polyol, in an amount ranging from 1% to 20% by weight with respect to the total weight of the composition, such as, for example, ranging from 3% to 15% by weight.

The aqueous phase may additionally comprise at least one stabilizing agent, such as, for example, sodium chloride, magnesium dichloride and magnesium sulphate.

The aqueous phase may also comprise at least one of any water-soluble or water-dispersible compound compatible with an aqueous phase, such as gelling agents, film-forming polymers, thickeners, surfactants and mixtures thereof.

In one embodiment of the present disclosure, the aqueous phase can be present in the emulsion in an amount ranging from 10% to 80% by weight with respect to the total weight of the emulsion, such as, for example, ranging from 15% to 60% by weight or ranging from 20% to 50% by weight.

The composition according to the present disclosure comprises an oily phase comprising particles.

According to one embodiment, the particles dispersed in the oily phase have a mean size by volume of less than or equal to 50 μm (for example, ranging from 1 to 50 μm), less than or equal to 30 μm (for example, ranging from 1 to 30 μm), less than or equal to 15 μm (for example, ranging from 1 to 15 μm), or less than or equal to 10 μm.

According to another embodiment, the particles dispersed in the oily phase may have a mean size by volume of less than or equal to 15 μm (for example, ranging from 1 to 15 μm), or less than or equal to 10 μm (for example, ranging from 1 to 10 μm).

According to a further embodiment, the particles dispersed in the oily phase may be chosen from wax powders, silica powders and mixtures thereof.

According another embodiment of the present disclosure, the particles dispersed in the oily phase can be chosen from wax powders.

The waxes present in the oily phase may be in the form of small particles having, for example, a mean size by volume ranging from 0.5 to 30 micrometres, such as ranging from 1 to 20 micrometres, or ranging from 5 to 10 micrometres, and subsequently denoted by the expression “microwaxes.”

Among microwaxes which may be used in the compositions according to the present disclosure, non-limiting mention may be made of carnauba microwaxes, such as those sold under the name of “MicroCare 350®” by Micro Powders, synthetic wax microwaxes, such as those sold under the name of “MicroEase 114S®” by Micro Powders, microwaxes composed of a mixture of carnauba wax and polyethylene wax, such as those sold under the names of “Micro Care 300®” and “310®” by Micro Powders, microwaxes composed of a mixture of carnauba wax and of synthetic wax, such as those sold under the name “Micro Care 325®” by Micro Powders, or polyethylene microwaxes, such as those sold under the names of “Micropoly 200®”, “220®”, “220L®” and “250S®” by Micro Powders.

According to another embodiment, the particles dispersed in the oily phase can be silica powders.

The silica powders may be, for example, the porous silica microspheres sold under the name “Silica Beads SB-700” by Myoshi or the “Sunsphere® H51,” “Sunsphere® H33” or “Sunsphere® H53” products sold by Asahi Glass; the amorphous silica microspheres coated with polydimethylsiloxane sold under the name “SA Sunsphere® H-33” and “SA Sunsphere® H-53” by Asahi Glass; or hydrophobic pyrogenic silicas, such as silicas having hydrophobic trimethylsiloxyl groups, for example sold under the references “Aerosil R812®” by Degussa or “Cab-O-Sil TS-530®” by Cabot, or silicas having hydrophobic dimethylsilyloxyl or polydimethylsiloxane groups, for example sold under the references “Aerosil R972®” or “Aerosil R974®” by Degussa or “Cab-O-Sil TS-610®” or “Cab-O-Sil TS-720®” by Cabot.

The particles dispersed in the oily phase of the emulsion according to the present disclosure may be a soft- focus filler as disclosed in European Patent No. EP 1 099 437 A.

The particles with a size of less than 50 μm dispersed in the oily phase may be present in the composition in an amount ranging from 0.1% to 15% by weight with respect to the total weight of the composition, such as, for example, ranging from 0.5% to 10% by weight or ranging from 0.5% to 8% by weight.

The emulsion according to the present disclosure can comprise, in its oily phase, from 0.1% to 15% by weight of waxes, with respect to the total weight of the composition. In one embodiment, the emulsion may comprise from 0.5% to 10% or from 0.5% to 8% by weight.

The oily phase of the composition of the present disclosure comprises at least one oil. The at least one oil may be chosen from volatile oils, non-volatile oils and mixtures thereof. In one embodiment, the composition comprises at least one volatile oil and at least one non-volatile oil.

In another embodiment of the present disclosure, the composition may comprise at least one volatile oil.

The term “volatile oil” is understood to mean, within the meaning of the present disclosure, any oil capable of evaporating on contact with the skin at ambient temperature and atmospheric pressure. The volatile oils of the invention may include volatile cosmetic oils which are liquid at ambient temperature and which have a non-zero vapor pressure, at ambient temperature and atmospheric pressure, ranging, for example, from 0.13 Pa to 40,000 Pa (0.001 to 300 mmHg) or ranging from 1.3 to 1,300 Pa (0.01 to 10 mmHg).

The at least one volatile oil can be chosen from volatile hydrocarbon oils, volatile silicone oils, and volatile fluorinated oils.

The term “hydrocarbon oil” is understood to mean, as used herein, an oil comprising mainly hydrogen and carbon atoms and optionally oxygen, nitrogen, sulphur and/or phosphorus atoms.

Among volatile hydrocarbon oils that can be used according to the present disclosure, non-limiting mention may be made of hydrocarbon oils having from 8 to 16 carbon atoms, including branched C₈-C₁₆ alkanes, such as C₈-C₁₆ isoalkanes of petroleum origin (also referred to as isoparaffins), such as isododecane (also known as 2,2,4,4,6-pentamethylheptane), isodecane, isohexadecane and, for example, the oils sold under the trade names of Isopars® or of Permethyls®.

Other volatile oils that may be used according to the present disclosure include volatile silicones, such as, for example, volatile linear or cyclic silicone oils, including those having a viscosity≦5 centistokes (5×10⁻⁶ m²/s) and having, for example, from 2 to 10 silicon atoms, such as from 2 to 7 silicon atoms, these silicones optionally comprising alkyl or alkoxy groups having from 1 to 10 carbon atoms. Further non-limiting mention may be made, as volatile silicone oils which may be used, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof.

The volatile fluorinated oil according to one embodiment does not have a flash point.

Among volatile fluorinated oils that may be used according to the present disclosure, non-limiting mention may be made of nonafluoroethoxybutane, nonafluoromethoxybutane, decafluoropentane, tetradecafluorohexane, dodecafluoropentane and mixtures thereof.

In one embodiment of the present disclosure, the composition comprises at least one volatile hydrocarbon oil, for example, a mixture of isododecane and of isohexadecane.

The at least one volatile oil may be present in the composition according to the present disclosure in an amount ranging from 1% to 60% by weight with respect to the total weight of the composition, such as, for example, ranging from 10% to 55% by weight, from 15% to 50% by weight, or from 20% to 50% by weight.

In addition, the composition according to the present disclosure may comprise at least one non-volatile oil.

As used herein, the term “non-volatile oil” is understood to mean an oil which remains on the skin at ambient temperature and atmospheric pressure for at least several hours and which has, for example, a vapor pressure of less than 0.13 Pa (0.01 mmHg).

Among the non-volatile oils that may be used according to the present disclosure, non-limiting mention may be made of hydrocarbon oils, such as those of animal or vegetable origin, silicone oils or mixtures thereof. As used herein, the term “hydrocarbon oil” is understood to mean an oil comprising mainly hydrogen and carbon atoms and optionally oxygen, nitrogen, sulphur and/or phosphorus atoms.

For example, the non-volatile oils may be chosen from non-volatile hydrocarbon oils, if appropriate fluorinated, and/or non-volatile silicone oils.

Non-limiting mention may be made, as non-volatile hydrocarbon oils, of:

hydrocarbon oils of animal origin,

hydrocarbon oils of vegetable origin, such as triglycerides composed of esters of fatty acids and of glycerol, the fatty acids of which can have various chain lengths from C₄ to C₂₄, it being possible for these chains to be linear or branched and saturated or unsaturated; these oils can be, for example, triglycerides of heptanoic acid or of octanoic acid, or alternatively wheat germ, sunflower, grape seed, sesame, maize, apricot, castor, shea, avocado, olive, soybean, sweet almond, palm, rapeseed, cottonseed, hazelnut, macadamia, jojoba, alfalfa, poppy, pumpkinseed, sesame, cucumber, blackcurrant seed, evening primrose, millet, barley, quinoa, rye, safflower, candlenut, passionflower or musk rose oil; shea butter; or triglycerides of caprylic/capric acids, such as those sold by Stéarineries Dubois or those sold under the names Miglyol 810®, 812® and 818® by Dynamit Nobel;

synthetic ethers having from 10 to 40 carbon atoms;

linear or branched hydrocarbons of mineral or synthetic origin, such as liquid petrolatum, polydecenes, hydrogenated polyisobutene, such as Parleam®, squalane, liquid paraffins and mixtures thereof;

synthetic esters, such as oils of formula R₁COOR₂ in which R₁ is the residue of a linear or branched fatty acid comprising from 1 to 40 carbon atoms and R₂ represents a hydrocarbon chain, such as a branched hydrocarbon chain, comprising from 1 to 40 carbon atoms, provided that R₁+R₂ is ≧10, such as, for example, Purcellin oil (cetearyl octanoate), isopropyl myristate, isopropyl palmitate, C₁₂ to C₁₅ alkyl benzoates, hexyl laurate, diisopropyl adipate, isononyl isononanoate, isodecyl neopentanoate, 2-ethylhexyl palmitate, isostearyl isostearate, 2-hexyldecyl laurate, 2-octyidecyl palmitate, 2-octyldodecyl myristate or heptanoates, octanoates, decanoates or ricinoleates of alcohols or of polyalcohols, such as propylene glycol dioctanoate; hydroxylated esters, such as isostearyl lactate, diisostearyl malate or 2-octyldodecyl lactate; esters of polyols and esters of pentaerythritol;

fatty alcohols which are liquid at ambient temperature with a branched and/or unsaturated carbon chain having from 12 to 26 carbon atoms, such as octyldodecanol, isostearyl alcohol, oleyl alcohol, 2-hexyldecanol, 2-butyloctanol and 2-undecylpentadecanol;

higher fatty acids, such as oleic acid, linoleic acid, linolenic acid and mixtures thereof.

Among the non-volatile silicone oils which can be used in the composition according to the present disclosure, non-limiting mention may be made of non-volatile polydimethylsiloxanes (PDMSs), polydimethylsiloxanes comprising pendant alkyl or alkoxy groups and/or alkyl or alkoxy groups at the end of the silicone chain, which groups each have from 2 to 24 carbon atoms, or phenylated silicones, such as phenyl trimethicones, phenyl dimethicones, phenyl(trimethylsiloxy)diphenylsiloxanes, diphenyl dimethicones or diphenyl(methyldiphenyl)trisiloxanes, and mixtures thereof.

In a further embodiment, the at least one non-volatile oil may be chosen from C₁₂-C₃₆ esters, such as those described above.

The at least one non-volatile oil may be present in the composition according to the present disclosure in an amount ranging from 1% to 60% by weight with respect to the total weight of the composition, such as, for example, from 5% to 50% by weight or from 5% to 40% by weight.

The oily phase of the emulsion may be present in an amount ranging from 1% to 60% by weight with respect to the total weight of the composition, such as, for example, from 5% to 50% by weight or from 5% to 40% by weight.

The emulsion according to the present disclosure may comprise at least one surfactant, such as, for example, a surfactant having an HLB (hydrophilic/lipophilic balance) suitable for the sense of the emulsion.

Among the surfactants which may be used according to the present disclosure and which are suitable for the preparation of an O/W emulsion, non-limiting mention may be made of those having an HLB of less than 7, including, for example, polyol fatty acid esters, such as sorbitol mono-, di-, tri- or sesquioleates or -stearates, glycerol mono-, di-, tri- or sesequioleates or -stearates, glycerol laurates or polyethylene glycol laurates; or alkyl or alkoxy dimethicone copolyols possessing a pendant alkyl or alkoxy chain or an alkyl or alkoxy chain at the end of the silicone backbone having, for example, from 6 to 22 carbon atoms.

Other surfactants which may be used according to the present disclosure for the preparation of an O/W emulsion, may include those having an HLB of greater than 7, such as polyethylene glycol fatty acid esters (polyethylene glycol monostearate or monolaurate); polyoxyethylenated fatty acid esters (stearate, oleate) of sorbitol; polyoxyethylenated alkyl (lauryl, cetyl, stearyl, octyl) ethers and dimethicone copolyols. In many embodiments according to the present disclosure, use may be made of at least one of any ionic (cationic or anionic) or amphoteric surfactant and any nonionic surfactant well known to a person skilled in the art.

The at least one surfactant may be present in the composition in an amount ranging from 0.3% to 10% by weight with respect to the total weight of the composition, such as, for example, from 1% to 5% by weight.

The emulsion according to the present disclosure may also comprise at least one additional filler other than the particles described above.

As used herein, the term “fillers” should be understood as meaning colorless or white and inorganic or synthetic particles of any shape which are insoluble in the medium of the composition, whatever the temperature at which the composition is manufactured.

The fillers may be inorganic or organic of any shape, platelet, spherical or oblong, whatever the crystallographic form (for example sheet, cubic, hexagonal, orthorhombic, and the like). Non-limiting mention may be made of talc, mica, silica, kaolin, powders formed of polyamide (Nylon®), of poly-β-alanine and of polyethylene, powders formed of tetrafluoroethylene polymers (Teflon®), lauroyllysine, starch, boron nitride, polymeric hollow microspheres, such as those of poly(vinylidene chloride/acrylonitrile), for example Expancel® (Nobel Industrie), or of acrylic acid copolymers, silicone resin microbeads (Tospearls® from Toshiba, for example), particles formed of polyorganosiloxane elastomers, precipitated calcium carbonate, magnesium carbonate, basic magnesium carbonate, hydroxyapatite, hollow silica microspheres, glass or ceramic microcapsules, metal soaps derived from organic carboxylic acids having from 8 to 22 carbon atoms, such as from 12 to 18 carbon atoms, for example zinc steareate, magnesium stearate, lithium stearate, zinc laurate or magnesium myristate, or poly(methyl methacrylate) particles.

The at least one additional filler may be present in the composition in an amount ranging from 0.1% to 25% by weight with respect to the total weight of the composition, such as, for example, from 1% to 20% by weight or from 5% to 15% by weight.

The emulsion according to the present disclosure may comprise at least one coloring material which may be chosen from water-soluble or fat-soluble dyes, pigments, pearlescent agents and mixtures thereof.

In the present disclosure, the term “pigments” is understood to mean white or colored and inorganic or organic particles which are insoluble in the liquid organic phase and which are intended to color and/or opacify the composition.

Pigments and pearlescent agents are particles in the solid state in the final composition. These solid particles are not dissolved in the final composition.

As used herein, the term “pearlescent agents” is understood to mean iridescent particles, such as those produced by certain shellfish in their shells or else synthesized, which are insoluble in the medium of the composition.

As used herein, the term “dyes” is understood to mean compounds, including organic compounds, which are soluble in fatty substances, such as oils, or in an aqueous/alcoholic phase.

The pigments may be inorganic or organic pigments. Among pigments that may be used according to the present disclosure, non-limiting mention may be made of metal oxides, such as iron oxides (for example, those which are yellow, red, brown or black in color), titanium dioxides, cerium oxide, zirconium oxide or chromium oxide; manganese violet, ultramarine blue, Prussian blue, ferric blue, bismuth oxychloride, mother-of-pearl, mica covered with titanium dioxide or with bismuth oxychloride, colored pearlescent pigments, such as titanium oxide-coated mica with iron oxides, titanium oxide-coated mica with, for example, ferric blue or chromium oxide, or titanium oxide-coated mica with an organic pigment of the abovementioned type, and pearlescent pigments based on bismuth oxychloride, and mixtures thereof.

In one embodiment, the at least one pigment can be chosen from iron oxides and titanium dioxide.

The at least one pigment may be treated with a hydrophobic agent in order to render them compatible with the organic phase of the composition. The hydrophobic treatment agent may be chosen from silicones, such as methicones, dimethicones or perfluoroalkylsilanes, fatty acids, such as stearic acid, metal soaps, such as aluminium dimyristate or the aluminium salt of hydrogenated tallow glutamate, perfluoroalkyl phosphates, perfluoroalkylsilanes, perfluoroalkylsilazanes, poly(hexafluoropropylene oxides), polyorganosiloxanes comprising perfluoroalkyl-perfluoropolyether groups, amino acids, N-acylated amino acids and/or the salts thereof, lecithin, isopropyl triisostearyl titanat and mixtures thereof.

The N-acylated amino acids may comprise an acyl group having from 8 to 22 carbon atoms, such as, for example, a 2-ethylhexanoyl, caproyl, lauroyl, myristoyl, palmitoyl, stearoyl or cocoyl group. The salts of these compounds can be chosen from aluminium, magnesium, calcium, zirconium, zinc, sodium and potassium salts. The amino acid may be chosen from, for example, lysine, glutamic acid and alanine.

The term “alkyl” in the abovementioned compounds denotes an alkyl group having from 1 to 30 carbon atoms, such as, for example, from 5 to 16 carbon atoms.

Among the hydrophobically-treated pigments that can be used according to the present disclosure, non-limiting mention may be made of those disclosed, for example, in European Patent Application No. EP 1 086 683 A.

The fat-soluble dyes may be chosen from, for example, Sudan red, D&C Red No. 17, D&C Green No. 6, β-carotene, soybean oil, Sudan brown, D&C Yellow No. 11, D&C Violet No. 2, D&C Orange No. 5, quinoline yellow, annatto and bromoacids.

The water-soluble dyes may be chosen from, for example, beetroot juice, methylene blue and caramel.

The at least one coloring material may be present in the composition in an amount ranging from 0.001% to 30% by weight with respect to the total weight of the composition, such as, for example, from 0.1% to 20% by weight or from 1 to 15% by weight.

The composition can be provided in the form of a water-in-oil emulsion, of an oil-in-water emulsion or of a multiple emulsion.

According to one embodiment of the present disclosure, the composition may be provided in the form of a water-in-oil emulsion or an oil-in-water emulsion.

The composition according to the present disclosure may comprise at least one other standard cosmetic ingredient which may be chosen from hydrophilic and lipophilic gelling and/or thickening agents; antioxidants; fragrances; preservatives; neutralizing agents; sunscreen agents; vitamins; moisturizing agents; self-tanning compounds; anti-wrinkle active principles; emollients; hydrophilic and lipophilic active principles; agents for combating free radicals; sequestering agents; and film-forming agents.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the invention as approximations, the numerical values set forth in the specific examples are reported as precisely as possible. Any numerical value, however, inherently contains certain errors necessarily resulting from the standard deviation found in its respective testing measurement.

The examples that follow are intended to illustrate the invention without, however, being limiting in nature.

### EXAMPLES

### Example 1

A foundation was prepared in the form of a water-in-oil emulsion having the following composition:

| | | |
|---|---|---|
| Silica sold under the name Sunsphere H51 | 4 | g |
| by Asahi Glass | | |
| Cetyl dimethicone copolyol (Abil EM 90 from | 2.7 | g |
| Goldschmidt) | | |
| Polyglyceryl-4 isostearate (Isolan GI 34 | 0.9 | g |
| from Goldschmidt) | | |
| Isostearyl palmitate | 6.0 | g |
| Cyclopentasiloxane | 30 | g |
| Hydrophobic coated iron oxides | 3.13 | g |
| Hydrophobic coated titanium oxides | 7.87 | g |
| Aqueous dispersion of colloidal silica | 17 | g |
| (40% AM) sold under the name Cosmo S 40 | | |
| by Catalysts & Chemicals | | |
| Water | q.s. for 100 | g |

The oils were mixed. The pigments were milled with cyclopentasiloxane. The milled preparation was added to the mixture of oils while stirring at ambient temperature.

The silica was subsequently dispersed with stirring.

The aqueous dispersion of silica and the water were then added while cooling, so as to retain an emulsification temperature close to ambient temperature.

The emulsion obtained comprised an aqueous dispersion of colloidal silica, and silica dispersed in the oily phase.

The foundation, applied to the skin, made it possible to obtain a make-up which efficiently masked the wrinkles and gave a firm and toned appearance to the skin.

### Example 2

A foundation was prepared in the form of a water-in-oil emulsion having the following composition:

| | | |
|---|---|---|
| Paraffin wax powder sold under the name | 4.0 | g |
| Microease 114S by Micropowders | | |
| Cetyl dimethicone copolyol (Abil EM 90 from | 2.7 | g |
| Goldschmidt) | | |
| Polyglyceryl-4 isostearate (Isolan GI 34 | 0.9 | g |
| from Goldschmidt) | | |
| Isostearyl palmitate | 6.0 | g |
| Cyclopentasiloxane | 20 | g |
| Hydrophobic coated iron oxides | 3.13 | g |
| Hydrophobic coated titanium oxides | 7.87 | g |
| Aqueous dispersion of colloidal silica | 17 | g |
| (40% AM) sold under the name Cosmo S 40 | | |
| by Catalysts & Chemicals | | |
| Water | q.s. for 100 | g |

The method of preparation was the same as that in Example 1.

The emulsion obtained comprised an aqueous dispersion of colloidal silica, and wax powder in the oily phase.

The foundation, applied to the skin, made it possible to obtain a make-up which efficiently masked the wrinkles and gave a firm and toned appearance to the skin.

### Example 3

In a first step, a microdispersion comprising 27% AM of carnauba wax was prepared, which microdispersion had the following composition:

| | | |
|---|---|---|
| Carnauba wax | 27 | g |
| Polyoxyethylenated (30 EO) glyceryl mono- | 6.75 | g |
| stearate (Tagat S from Goldschmidt) | | |
| Ethanol | 10 | g |
| Water | q.s. for 100 | g |

The wax and the surfactant were heated to 90° C. while homogenizing the mixture with moderate stirring. The water, heated to 90° C., was then incorporated while continuing to stir. The mixture was cooled to ambient temperature and the ethanol was added, in order to obtain a wax microdispersion having a mean particle diameter of approximately 170 nm.

A foundation was subsequently prepared in the form of an oil-in-water emulsion comprising the wax microdispersion prepared above and having the following composition:

| | | |
|---|---|---|
| Polydimethylsiloxane (Dow Corning | 9.00 | g |
| Fluid 200 5 cst from Dow Corning) | | |
| Caprylic/capric triglycerides (Myritol | 10.45 | g |
| from Cognis) | | |
| Isodecyl neopentanoate | 6.0 | g |
| Silica microspheres (Sunsphere H51 from | 4.0 | g |
| Dohkaï Chemical Industries) | | |
| Paraffin wax powder (Microease 114S from | 1.0 | g |
| Micro Powders) | | |
| Carnauba wax microdispersion in water | 10 | g |
| comprising 27% AM of wax | | |
| Dimethicone copolyol (Abil Care 85 from | 3.45 | g |
| Goldschmidt) | | |
| Gelling agent (Sepigel 305 from Seppic) | 0.20 | g |
| Glycerol | 5.0 | g |
| Butylene glycol | 4.0 | g |
| Oxyethylene sorbitan monooleate | 10 | g |
| (Tween 80V Pharma from Uniquema) | | |
| Magnesium aluminium silicate | 0.50 | g |
| (Veegum granules from Vanderbilt) | | |
| Talc | 3.00 | g |
| Titanium dioxide | 8.75 | g |
| Iron oxide | 2.25 | g |
| Preservatives | q.s. | |
| Water | q.s. for 100 | g |

The preservatives and the moisturizing agents were introduced into the water. The pre-prepared wax microdispersion was added thereto at ambient temperature.

The pigments were milled in a water/magnesium aluminium silicate mixture and then the milled preparation was added to the aqueous phase.

The oils, the surfactants and the microwaxes were mixed.

The emulsion was prepared at ambient temperature and then the talc, the silica microspheres and the gelling agent were added.

The emulsion obtained comprised an aqueous dispersion of waxes, and wax powder in the oily phase.

The foundation, applied to the skin, made it possible to obtain a make-up which efficiently masked the wrinkles and gave a firm and toned appearance to the skin.

What is claimed is:

1. A cosmetic composition for making up or caring for the skin in the form of an emulsion comprising an aqueous phase and an oily phase, wherein the aqueous phase comprises a dispersion of at least one wax particles with a mean size by volume of less than or equal to 1 μm and the oily phase comprises a dispersion of particles with a mean size by volume of less than or equal to 15 μm.

2. The cosmetic composition according to claim 1, wherein the dispersion of wax particles in the aqueous phase comprises wax particles having a mean size by volume of less than or equal to 0.5 μm.

3. The cosmetic composition according to claim 1, wherein the at least one wax is chosen from carnauba wax, candelilla wax, ouricury wax, Japan wax, cork fiber, sugarcane wax, ceresin, paraffin waxes, lignite wax, microcrystalline waxes, lanolin wax, montan wax, ozokerite, hydrogenated oils, waxed obtained by Fischer-Tropsch synthesis, fatty acid esters, glycerides which are solid at 45° C., silicone waxes, and fatty acids.

4. The cosmetic composition according to claim 3, wherein the at least one hydrogenated oil is chosen from hydrogenated jojoba oil and copolymerization of ethylene.

5. The cosmetic composition according to claim 3, wherein the at least one silicone wax is chosen from alkyl, alkoxy, and ester derivatives of poly(di)methylsiloxane which are solid at 45° C. and have from 10 to 45 carbon atoms.

6. The cosmetic composition according to claim 3, wherein the at least one fatty acid is chosen from stearic acid, myristic acid and behenic acid.

7. The cosmetic composition according to claim 1, wherein the at least one wax particles dispersed in the aqueous phase are present in an amount ranging from 0.01% to 15% by weight, relative to the total weight of the composition.

8. The cosmetic composition according to claim 7, wherein the at least one wax particles dispersed in the aqueous phase are present in an amount ranging from 1% to 7% by weight, relative to the total weight of the composition.

9. The cosmetic composition according to claim 1, wherein the particles dispersed in the oily phase are present in an amount ranging from 0.1% to 15% by weight, relative to the total weight of the composition.

10. The cosmetic composition according to claim 9, wherein the particles dispersed in the oily phase are present in an amount ranging from 0.5% to 8% by weight, relative to the total weight of the composition.

11. The cosmetic composition according to claim 1, further comprising at least one oil.

12. The cosmetic composition according to claim 1, further comprising at least one volatile oil.

13. The cosmetic composition according to claim 12, wherein the at least one volatile oil is chosen from volatile hydrocarbon oils having from 8 to 16 carbon atoms; linear and cyclic silicone oils having from 2 to 7 silicon atoms, wherein the silicone oils optionally comprise alkyl or alkoxy groups having from 1 to 10 carbon atoms; and volatile fluorinated oils.

14. The cosmetic composition according to claim 12, wherein the at least one volatile oil is chosen from isododecane, isodecane, isohexadecane, octamethylcyclotetrasiloxane, decamethylcyclopentasiloxane, dodecamethylcyclohexasiloxane, heptamethylhexyltrisiloxane, heptamethyloctyltrisiloxane, hexamethyidisiloxane, octamethyltrisiloxane, decamethyltetrasiloxane and dodecamethylpentasiloxane.

15. The cosmetic composition according to claim 12, wherein the at least one volatile oil is present in an amount ranging from 1% to 60% by weight, relative to the total weight of the composition.

16. The cosmetic composition according to claim 15, wherein the at least one volatile oil is present in an amount ranging from 20% to 50% by weight, relative to the total weight of the composition.

17. The cosmetic composition according to claim 1, further comprising at least one non-volatile oil.

18. The cosmetic composition according to claim 17, wherein the at least one non-volatile oil is chosen from non-volatile silicone oils, non-volatile hydrocarbon oils, and non-volatile fluorinated oils.

19. The cosmetic composition according to claim 18, wherein the at least one non-volatile oil is a non-volatile silicone oil.

20. The cosmetic composition according to claim 19, wherein the at least one non-volatile oil is chosen from non-volatile polydimethylsiloxanes; polydimethylsiloxanes comprising pendant alkyl, alkoxy or phenyl groups or alkyl, alkoxy or phenyl groups at the end of the silicone chain, which groups have from 2 to 24 carbon atoms; phenylated silicones; polysiloxanes modified by fatty acids, fatty alcohols or polyoxyalkylenes; aminated silicones; silicones comprising hydroxyl groups; and fluorosilicones comprising a pendant fluorinated group or a fluorinated group at the end of the silicone chain having from 1 to 12 carbon atoms, all or part of the hydrogens of which are substituted by fluorine atoms.

21. The cosmetic composition according to claim 17, wherein the at least one non-volatile oil is chosen from liquid paraffin, squalane, hydrogenated polyisobutylene (Parleam oil), perhydrosqualene, mink oil, turtle oil, soybean oil, sweet almond oil, calophyllum oil, palm oil, grape seed oil, sesame oil, maize oil, arara oil, rapeseed oil, sunflower oil, cottonseed oil, apricot oil, castor oil, avocado oil, jojoba oil, olive oil, cereal germ oil, C₁₂-C₃₆ fatty esters, higher C₁₄-C₂₂ fatty acids, and higher C₁₆-C₂₂ fatty alcohols.

22. The cosmetic composition according to claim 17, wherein the at least one non-volatile oil is present in an amount ranging from 1% to 60% by weight, relative to the total weight of the composition.

23. The cosmetic composition according to claim 22, wherein the at least one non-volatile oil is present in an amount ranging from 5% to 50% by weight, relative to the total weight of the composition.

24. The cosmetic composition according to claim 1, wherein the composition comprises water in an amount ranging from 10% to 80% by weight, relative to the total weight of the composition.

25. The cosmetic composition according to claim 24, wherein the composition comprises water in an amount ranging from 20% to 50% by weight, relative to the total weight of the composition.

26. The cosmetic composition according to claim 1, wherein the aqueous phase further comprises at least one solvent chosen from ethanol, isopropanol, glycerol, propylene glycol, butylene glycol, hexylene glycol, dipropylene glycol, diethylene glycol, mono-, di- and/or tripropylene glycol (C₁-C₄)alkyl ethers, and mono-, di- and/or triethylene glycol (C₁-C₄)alkyl ethers.

27. The cosmetic composition according to claim 1, wherein the aqueous phase is present in an amount ranging from 10% to 80% by weight, relative to the total weight of the composition.

28. The cosmetic composition according to claim 27, wherein the aqueous phase is present in an amount ranging from 20% to 50% by weight, relative to the total weight of the composition.

29. The cosmetic composition according to claim 1, further comprising at least one surfactant.

30. The cosmetic composition according to claim 1, wherein the composition is provided in a form chosen from an oil-in-water emulsion and a water-in-oil emulsion.

31. The cosmetic composition according to claim 1, further comprising at least one additional filler other than the particles dispersed in the aqueous phase and in the oily phase.

32. The cosmetic composition according to claim 31, wherein the at least one additional filler is chosen from talc, mica, silica, kaolin, polyamide powders, poly-β-alanine powders, polyethylene powders, powders formed of tetrafluoroethylene polymers, lauroyllysine, starch, boron nitride, hollow microspheres of poly(vinylidene chloride/acrylonitrile), hollow microspheres of acrylic acid copolymers, silicone resin microbeads, particles formed of polyorganosiloxane elastomers, precipitated calcium carbonate, magnesium carbonate, basic magnesium carbonate, hydroxyapatite, hollow silica microspheres, porous silicas, ceramic microcapsules, and metal soaps derived from organic carboxylic acids having from 8 to 22 carbon atoms.

33. The cosmetic composition according to claim 31, wherein the at least one additional filler is present in an amount ranging from 0.1% to 25% by weight, relative to the total weight of the composition.

34. The cosmetic composition according to claim 33, wherein the at least one additional filler is present in an amount ranging from 5% to 15% by weight, relative to the total weight of the composition.

35. The cosmetic composition according to claim 1, further comprising at least one coloring material.

36. The cosmetic composition according to claim 35, wherein the at least one coloring material is chosen from pigments, pearlescent agents, and dyes.

37. The cosmetic composition according to claim 35, wherein the at least one coloring material is present in an amount ranging from 0.001% to 30% by weight, relative to the total weight of the composition.

38. The cosmetic composition according to claim 35, wherein the at least one coloring material is present in an amount ranging from 1% to 15% by weight, relative to the total weight of the composition.

39. The cosmetic composition according to claim 1, further comprising at least one cosmetic ingredient chosen from hydrophilic and lipophilic gelling and/or thickening agents; antioxidants; fragrances; preservatives; neutralizing agents; sunscreen agents; vitamins; moisturizing agents; self-tanning compounds; anti-wrinkle active principles; emollients; hydrophilic and lipophilic active principles; agents for combating free radicals; sequestering agents; and film-forming agents.

40. A cosmetic composition for making up or caring for the skin in the form of an emulsion comprising an aqueous phase and an oily phase, wherein the aqueous phase comprises a dispersion of at least one compound in the form of particles with a mean size by volume of less than or equal to 1 μm, and the oily phase comprises a dispersion of particles of the same at least one compound with a mean size by volume of less than or equal to 50 μm.

41. The cosmetic composition according to claim 40, wherein the particles with a mean size by volume of less than or equal to 1 μm dispersed in the aqueous phase are chosen from waxes, inorganic compounds and mixtures thereof.

42. The cosmetic composition according to claim 41, wherein the inorganic compounds are chosen from silicas, aluminas and mixtures thereof.

43. A cosmetic composition for making up or caring for the skin in the form of an emulsion comprising an aqueous phase and an oily phase, wherein the aqueous phase comprises a dispersion of particles with a mean size by volume of less than or equal to 1 μm chosen from waxes, silica particles and mixtures thereof, and the oily phase comprises a dispersion of particles chosen from wax powders, silica powders and mixtures thereof.

44. The cosmetic composition according to claim 43, wherein the aqueous phase comprises a dispersion of wax particles with a mean size by volume of less than or equal to 1 μm and the oily phase comprises a dispersion of wax powder.

45. The cosmetic composition according to claim 44, wherein the dispersion of wax particles in the aqueous phase comprises wax particles having a mean size by volume of less than or equal to 0.5 μm.

46. The cosmetic composition according claim 43, wherein the wax is chosen from carnauba wax, candelilla wax, ouricury wax, Japan wax, cork fiber, sugarcane wax, ceresin, paraffin waxes, lignite waxes, microcrystalline waxes, lanolin wax, montan wax, ozokerites, hydrogenated oils, waxes obtained by Fischer-Tropsch synthesis, fatty acid ester, glycerides which are solid at 45° C., silicone waxes, fatty acids, and mixtures thereof.

47. The cosmetic composition according to claim 46, wherein the hydrogenated oils can be chosen from hydrogenated jojoba oil and copolymerization of ethylene.

48. The cosmetic composition according to claim 46, wherein the silicone waxes are chosen from alkyl, alkoxy, and ester derivatives of poly(di)methylsiloxane which are solid at 45° C. and have from 10 to 45 carbon atoms, and mixtures thereof.

49. The cosmetic composition according to claim 46, wherein the fatty acids are chosen from stearic acid, myristic acid and behenic acid.

50. The cosmetic composition according to claim 43, wherein the wax dispersed in the aqueous phase is present in an amount ranging from 0.01% to 15% by weight, relative to the total weight of the composition.

51. The cosmetic composition according to claim 50, wherein the wax dispersed in the aqueous phase is present in an amount ranging from 1% to 7% by weight, relative to the total weight of the composition.

52. The cosmetic composition according to claim 43, wherein the particles dispersed in the oily phase have a mean size by volume of less than or equal to 30 μm.

53. The cosmetic composition according to claim 52, wherein the particles dispersed in the oily phase have a mean size by volume of less than or equal to 10 μm.

54. A cosmetic composition for making up or caring for the skin in the form of an emulsion comprising an aqueous phase and an oily phase, wherein the aqueous phase comprises a dispersion of particles of at least one material possessing a tightening effect with a mean size by volume of less than or equal to 1 μm, and the oily phase comprises a dispersion of particles with a mean size by volume of less than or equal to 15 μm.

55. A non-therapeutic process for making up or caring for the skin comprising applying to the skin a cosmetic composition comprising an emulsion comprising a dispersion of wax particles with a mean size by volume of less than or equal to 1 μm in the aqueous phase and an oily phase comprising a dispersion of particles with a mean size by volume of less than or equal to 15 μm.

56. A method for softening wrinkles and/or giving a firm and toned appearance to skin, comprising applying to the skin a composition comprising an emulsion comprising an aqueous phase and an oily phase, wherein the aqueous phase comprises a dispersion of wax particles with a mean size by volume of less than or equal to 1 μm, and the oily phase comprises a dispersion of particles with a mean size by volume of less than or equal to 15 μm.

57. A method for producing a make-up for the skin which softens wrinkles and/or which confers a firm and toned appearance on the skin, comprising:
preparing an emulsion comprising an aqueous phase and an oily phase, wherein the aqueous phase comprises a dispersion of wax particles with a mean size by volume of less than or equal to 1 μm, and the oily phase comprises a dispersion of particles with a mean size by volume of less than or equal to 15 μm.
